(19) United States
(12) Patent Application Publication
(10) Pub. No.: US 2018/0304093 A1
(43) Pub. Date: Oct. 25, 2018

(54) LASER TREATMENT DEVICE USING ULTRASONIC PROBE

(71) Applicant: Moonho SON, Daejeon (KR)

(72) Inventor: Moonho SON, Daejeon (KR)

(21) Appl. No.: 15/767,996

(22) PCT Filed: Oct. 14, 2016
(86) PCT No.: PCT/KR2016/011544

§ 371 Date: Apr. 12, 2018

(30) Foreign Application Priority Data
Oct. 16, 2015 (KR) 10-2015-0144443

(51) Int. Cl. A61N 5/06; A61B 8/08; A61B 8/00

(52) U.S. Cl. CPC A61N 5/0616; A61N 2005/067; A61B 8/4209; A61B 8/0833

(57) ABSTRACT

The present invention detects a lesion site using an ultrasonic probe and irradiates a laser on the detected lesion site to treat the lesion site, thereby having advantages of saving operation time, focusing a laser only on an exact lesion site so as to be treated, reducing an error with respect to the lesion site because even if an irradiation angle of the laser irradiated from a first irradiation part is changed, a target point reached by the laser remains the same.

### CROSS-REFERENCE TO RELATED APPLICATIONS

This patent application is a national-stage application of International Patent Application No. PCT/KR2016/011544, filed on Oct. 14, 2016, which claims priority under 35 U.S.C. § 119 to Korean Patent Application No. 10-2015-0144443, filed on Oct. 16, 2015, in the Korean Intellectual Property Office, the disclosure of which is incorporated by reference herein in its entirety.

### TECHNICAL FIELD

The present invention relates to a laser treatment device using an ultrasonic probe, and more specifically, to a laser treatment device capable of detecting a lesion using an ultrasonic probe and radiating laser beams to the lesion to treat the lesion.

### DISCUSSION OF RELATED ART

There are an increasing number of patients who suffer from muscle injury or pain due to strenuous exercise.

For the diagnosis of muscle injuries or myomas, orthopedic and pain clinics have been carrying out a biometric test on the patient's intracorporeal tissues using musculoskeletal ultrasound imaging. The following is a conventional way for diagnosing the patient's lesion using an ultrasonic probe.

If the operator is right-handed, he examines the lesion while moving the ultrasonic probe with the ultrasonic probe in his right hand and then grabs the ultrasonic probe in his left hand and an injection unit in his right hand and injects the serum into the lesion or performs paracentesis.

However, such a conventional way may frequently dislocate the ultrasonic probe by the grab changes from the right to left hand, changing the image produced based on ultrasonic echoes and hence forcing the operator to recheck the lesion.

To address such issues, Korean Patent No. 10-1409836, patented on Jun. 13, 2014, discloses an ultrasonic probe-integrated injector. FIG. 1 is a cross-sectional view illustrating an ultrasonic probe-integrated injector according to the prior art.

The ultrasonic probe-integrated injector includes a main body 10, a support 11, an injection unit 12, an angling means 13, a back-and-forth moving means 14, a manipulating unit 15, an ultrasonic probe 16, an angle detecting unit 17, and a control means 18.

According to the prior art, the main body 10 is shaped as a gun and includes a receiving space 10a for installing the other components while supporting the support 11, so that the injection unit 12 may be moved back and forth while being supported on one side of the support 11. A mode switch 10b and a driving switch 10c are provided on one side of the main body 10. The mode switch 10b may enable selection of any one of an angle adjusting mode, an injection unit moving mode, and a piston driving mode. Where one mode is selected by the mode switch 10b, the driving switch 10c enables the selected mode to be driven. If the driving switch 10c turns on with the angle adjusting mode selected by the mode switch 10b, a rotational force providing means 13a of the angle adjusting means 13 may be driven. When the injection unit moving mode is selected by the mode switch 10b and the driving switch 10c is turned on, a cylinder load 15a of the manipulating unit 15 is moved back and forth.

However, the conventional art fails to give a hard support, thus subjecting the injection unit to a momentary change in angle when inserted. Further, the injection unit may be angled only up and down, causing it difficult to insert the injection unit into the legion that might be positioned on the side or back.

To address the problems, Korean Patent No. 10-1508919, patented on Mar. 31, 2015, discloses an injector with a both hand-supportable ultrasonic probe. FIG. 2 is a perspective view illustrating an injector with a both hand-supportable ultrasonic probe according to the prior art.

The injector with a both hand-supportable ultrasonic probe includes an ultrasonic probe 120, a probe support 21, a side part 22, an adjusting body 23, an injection angling means 24, a height adjusting means 25, a rotational position adjusting means 26, and an injection unit fastening means 27.

According to the prior art, the ultrasonic probe 120 is combined with the injection unit 28. In this position, the legion is detected by the ultrasonic probe 120, and the needle of the injection unit 28 is precisely inserted to the lesion. The ultrasonic probe 120 and the injection unit 28 are supported by both hands, so that the needle of the injection unit 28 is prevented from changing in its insertion angle and may easily be inserted to the lesion in all directions.

According to the prior art, the angle at which the needle of the injection unit 28 is adjusted by the injection angle adjusting means 24. Adjusting the angle of insertion of the needle requires that the injection angle adjusting means 24 be fastened by manipulating an angle adjusting screw 26a in the positioned adjusted by the injection angle adjusting means 24, causing an error in the angle of insertion of the needle.

Further, according to the prior art, the depth to which the needle of the injection unit 28 is inserted is adjusted by the height adjusting means 25, and to do so, a height adjusting knob 25a needs to be rotated with the ultrasonic probe 120 or probe support 21 grabbed in one hand, forcing both hands to be used.

Moreover, the conventional art requires that the needle of the injection unit 28 be inserted to the lesion detected by the ultrasonic probe 120 and is thus difficulty to apply to patients that fear syringe needles and to use only detected lesions.

### SUMMARY

The present invention has been created to address the problems with the prior art, and an objective of the present invention is to provide a laser treatment device capable of detecting a lesion using an ultrasonic probe and radiating a laser beam to the lesion to treat the lesion.

An objective of the present invention is to provide a laser treatment device using an ultrasonic probe in which a laser beam radiated from a first radiating unit, although subjected to a change in radiation angle, may reach the target.

An objective of the present invention is to provide a laser treatment device using an ultrasonic probe in which the first radiating unit may simply be adjusted by either hand for its laser radiation angle and depth.

An objective of the present invention is to provide a laser treatment device using a ultrasonic probe in which while the depth of laser radiation is adjusted by a conveying rail and adjusting gears, the adjusting gears are spaced apart from the conveying rail by an elastic body provided between the conveying rail and the adjusting gears so that the depth of laser radiation would not be changed unless the adjusting gears are pressed hard.

To achieve the foregoing objectives, according to the present invention, a laser treatment device using an ultrasonic probe comprises an ultrasonic probe including a housing, a knob, a body connected to the knob, and a dispersing unit connected to the body, the body rotatably installed inside the housing, an elevator provided on an outer circumference of the housing to slide up or down, a first support having an end connected to a side of the elevator and shaped as an arc to have the same central axis along a lengthwise direction thereof, and a first radiating unit movably installed along a lengthwise direction of the first support to radiate laser beams.

The elevator includes a conveying rail formed between an upper protrusion and a lower protrusion formed on an outside of the housing, an opening formed in a side of the elevator to correspond to the conveying rail, and adjusting gears provided in the opening to face the conveying rail and rotating to slide the elevator along the conveying rail.

According to another embodiment, the elevator is combined with guide rails formed between the upper protrusion and lower protrusion of the housing to slide along the guide rails. In this case, at least one of the guide rails may be marked with numerical gradations for determining the height of the elevator as the elevator slides.

The first radiating unit includes a plurality of laser modules for radiating laser beams and a convex lens focusing the laser beams radiated from the plurality of laser modules.

The number of the plurality of laser modules is at least three or more, and the plurality of laser modules are arranged at even intervals and sequentially output and radiate the laser beams.

The present invention may detect a lesion using the ultrasonic probe and treat the legion by laser radiation, allowing for time savings for surgery. The present invention enables precise and intensive focusing only onto the legion upon treatment.

The present invention allows laser beams from the first radiating unit to reach the lesion despite a change in the angle of laser radiation, reducing an error in the lesion.

The present invention enables simple, one-hand adjustment on the angle and depth of laser radiation from the first radiating unit, and allows the depth and angle, after adjusted, to automatically be fixed, enhancing the work efficiency.

### BRIEF DESCRIPTION OF THE DRAWINGS

FIG. 1 is a cross-sectional view illustrating an ultrasonic probe-integrated injector according to the prior art;

FIG. 2 is a perspective view illustrating an injector with a both hand-supportable ultrasonic probe according to the prior art;

FIG. 3 is a front perspective view illustrating a laser treatment device using an ultrasonic probe according to the present invention;

FIG. 4 is a rear perspective view illustrating a laser treatment device using an ultrasonic probe according to the present invention;

FIG. 5 is a perspective view illustrating an example of rotating an ultrasonic probe in a laser treatment device using the ultrasonic probe according to the present invention;

FIG. 6 is a view illustrating a slide of an elevator of a laser treatment device using an ultrasonic probe according to the present invention;

FIG. 7 is a view illustrating a first support and a first radiating unit of a laser treatment device using an ultrasonic probe according to the present invention;

FIG. 8 is a perspective view illustrating a combination of a first support and a first radiating unit of a laser treatment device using an ultrasonic probe according to the present invention;

FIG. 9 is a view illustrating a laser module and a convex lens of a laser treatment device using an ultrasonic probe according to the present invention;

FIG. 10 is a view illustrating a second support and a second radiating unit of a laser treatment device using an ultrasonic probe according to the present invention;

FIG. 11 is a perspective view illustrating a guide rail of a laser treatment device using an ultrasonic probe according to the present invention;

FIG. 12 is a perspective view illustrating a conveying rail, an opening, and an adjusting gear of a laser treatment device using an ultrasonic probe according to the present invention;

FIGS. 13a and 13b are perspective views illustrating elastic bodies of a laser treatment device using an ultrasonic probe according to the present invention;

FIG. 14 is a perspective view illustrating another embodiment of an elevator and a first radiating unit of a laser treatment device using an ultrasonic probe according to the present invention: and

FIG. 15 is a view illustrating an example of remotely controlling a laser treatment device using an ultrasonic probe according to the present invention.

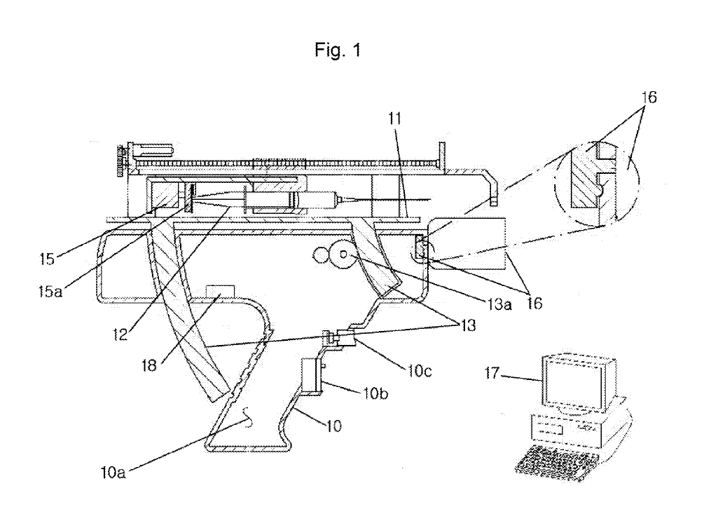

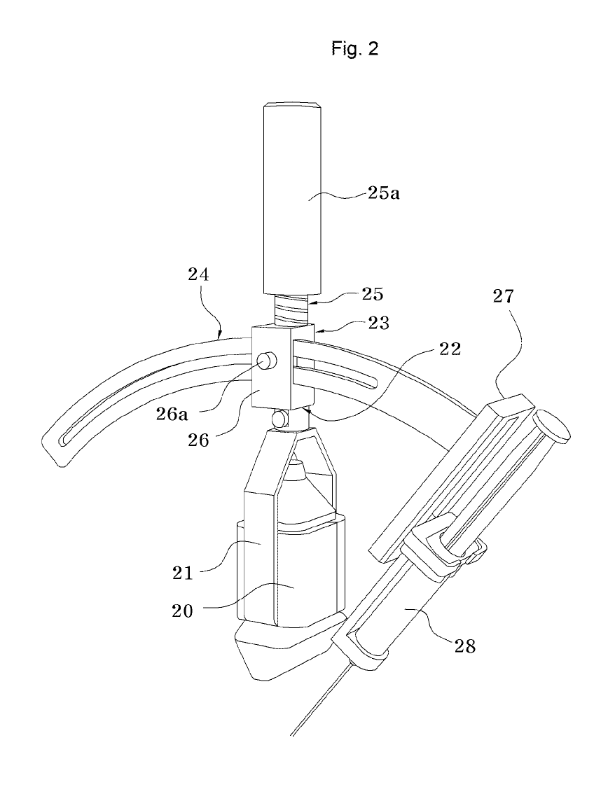

* Description of reference numbers*

| 100: laser treatment device using ultrasonic probe | |
|---|---|
| 110: housing | 111: upper protrusion |
| 112: lower protrusion | 113: guide rail |
| 114: conveying rail | 115: graduated unit |
| 120: ultrasonic probe | 121: knob |
| 122: body | 123: dispersing unit |
| 130: elevator | 131: opening |
| 132: adjusting gear | 133: elastic body |
| 134: hole | 140: first support |
| 141: fastening hole | 140a: second support |
| 150: first radiating unit | 151: laser module |
| 152: convex lens | 153: fastener |
| 154: elastic body | 155: moving wheel |
| 156: radiating module | 157: moving module |
| 150a: second radiating unit | 160: controller |

### DESCRIPTION OF EXEMPLARY EMBODIMENTS

Hereinafter, preferred embodiments of the present invention are described in detail with reference to the accompanying drawings.

The present invention relates to a laser treatment device capable of detecting a lesion using an ultrasonic probe and radiating a laser beam to the lesion to treat the lesion.

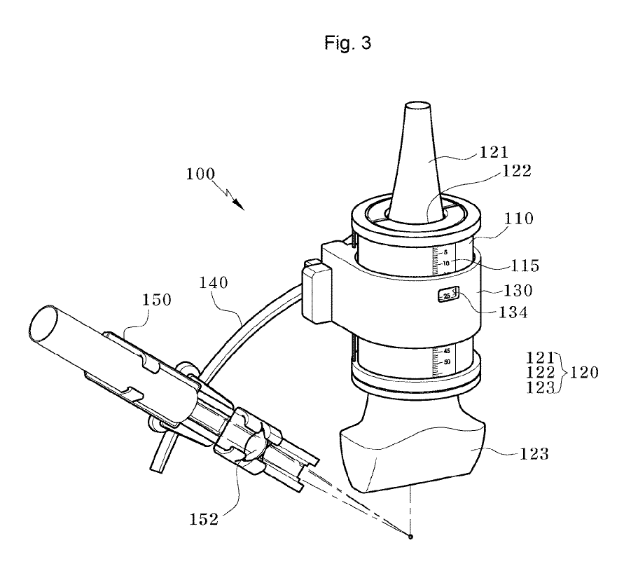

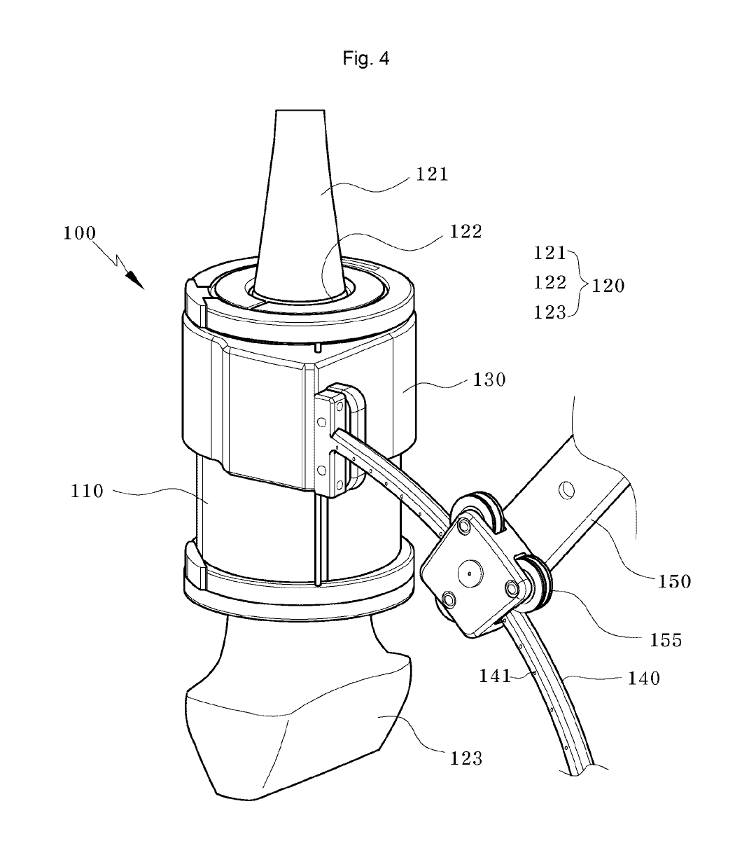

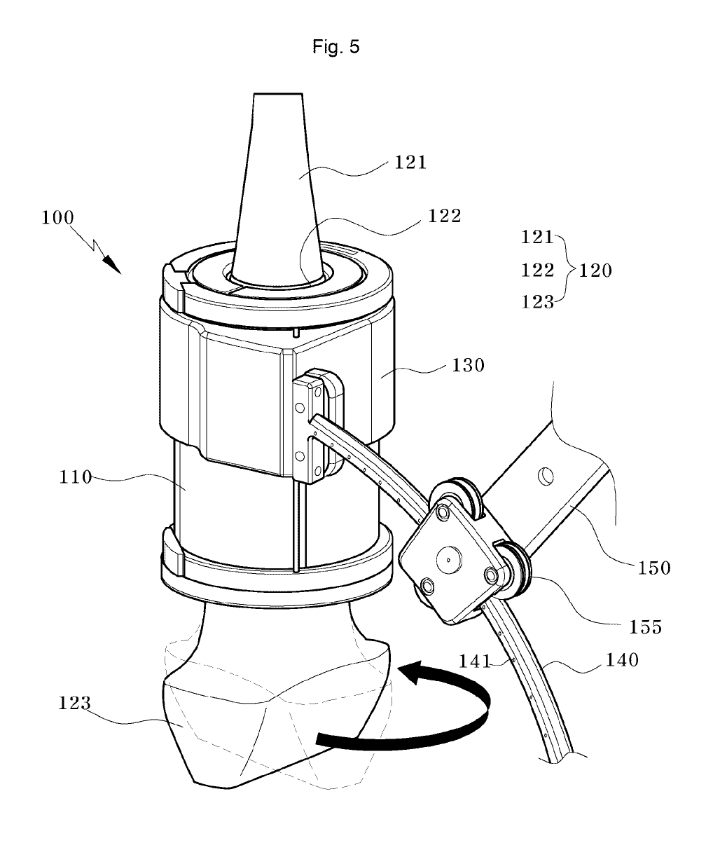

FIG. 3 is a front perspective view illustrating a laser treatment device using an ultrasonic probe according to the present invention. FIG. 4 is a rear perspective view illustrating a laser treatment device using an ultrasonic probe according to the present invention. FIG. 5 is a perspective view illustrating an example of rotating an ultrasonic probe in a laser treatment device using the ultrasonic probe according to the present invention.

Referring to FIGS. 3 and 4, according to the present invention, a laser treatment device using an ultrasonic probe includes a housing 110, an ultrasonic probe 120, an elevator 130, a first support 140, and a first radiating unit 150.

The housing 110 is intended for fastening and supporting the ultrasonic probe 120 surrounds and fastens the ultrasonic probe 120. Thus, the ultrasonic probe 120 may detect a lesion while being supported stably.

The ultrasonic probe 120 detects a lesion through an image produced based on ultrasonic echos. The ultrasonic probe 120 includes a knob 121, a body 122 connected with the knob 121, and a dispersing unit 123 connected with the body 122. The body 122 is rotatably installed inside the housing 110. Referring to FIG. 5, the dispersing unit 123 of the ultrasonic probe 120 may be configured to be rotatable with respect to the body 122.

According to the present invention, the ultrasonic probe 120 may be connected with a monitor (not shown) and may transmit image signals to the monitor to be displayed on the monitor, allowing the lesion to be recognized with the naked eye and detected.

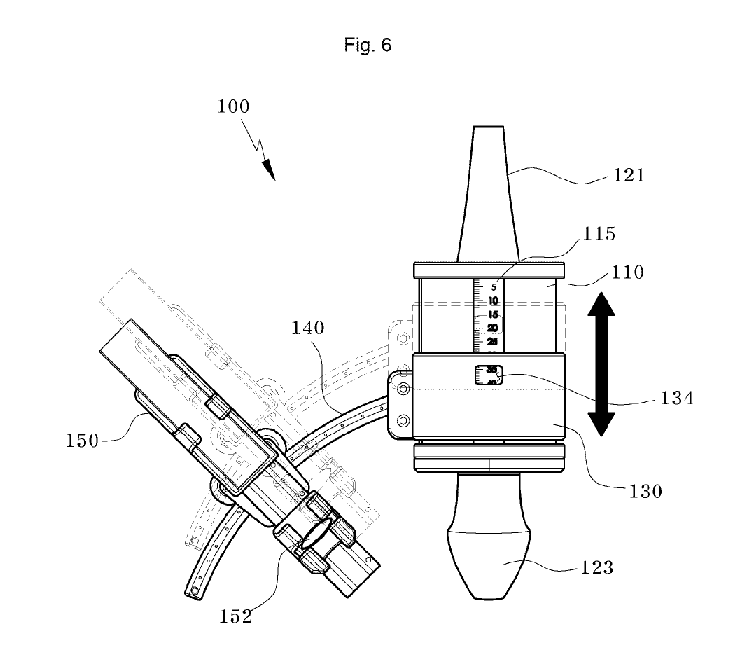

FIG. 6 is a view illustrating a slide of an elevator of a laser treatment device using an ultrasonic probe according to the present invention.

Referring to FIG. 6, the elevator 130 is installed on the outer circumference of the housing 110 to slide up or down.

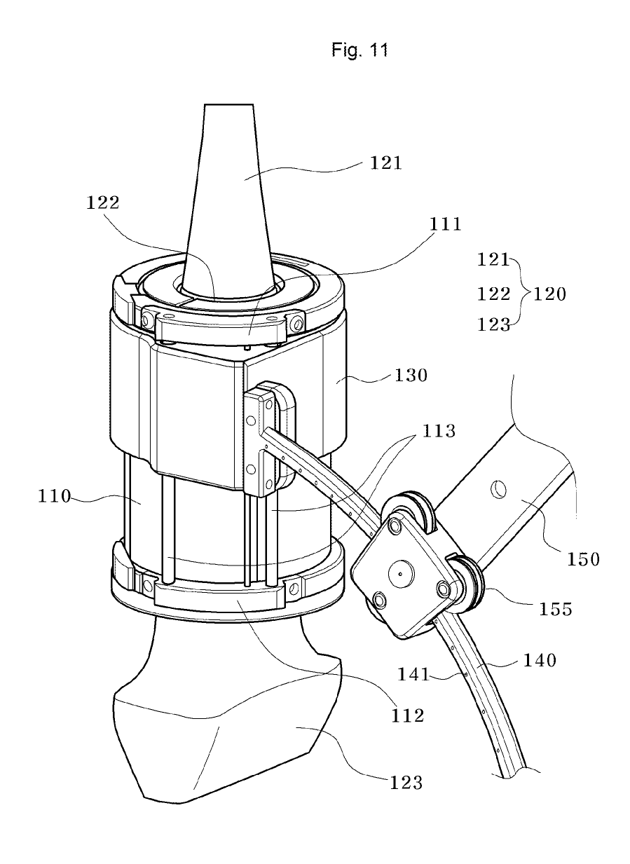

The elevator 130 includes a conveying rail 114 formed between an upper protrusion 111 and a lower protrusion 112 formed on the outside of the housing 110, a opening 131 formed in one side of the elevator 130 to correspond to the conveying rail 114, and adjusting gears 132 provided in the opening 131 to face the conveying rail 114 and rotating to allow the elevator 130 to slide along the conveying rail 114. The elevator 130 may further include guide rails 113 for a stable slide. Embodiments of the conveying rail 114, the opening 131, the adjusting gears 132, and the guide rails 113 are described below with reference to FIGS. 11 to 13b.

According to the present invention, a graduated unit 115 marked with numerical measurements may be provided on a side of the housing 110, and a hole 134 corresponding to the graduated unit 115 in one side of the elevator 130. The graduated unit 115 and the hole 134 may be provided to show the depth to which a laser beam from the first radiating unit 150 is radiated as the elevator 130 slides, allowing for an easier check on the depth of the laser radiation.

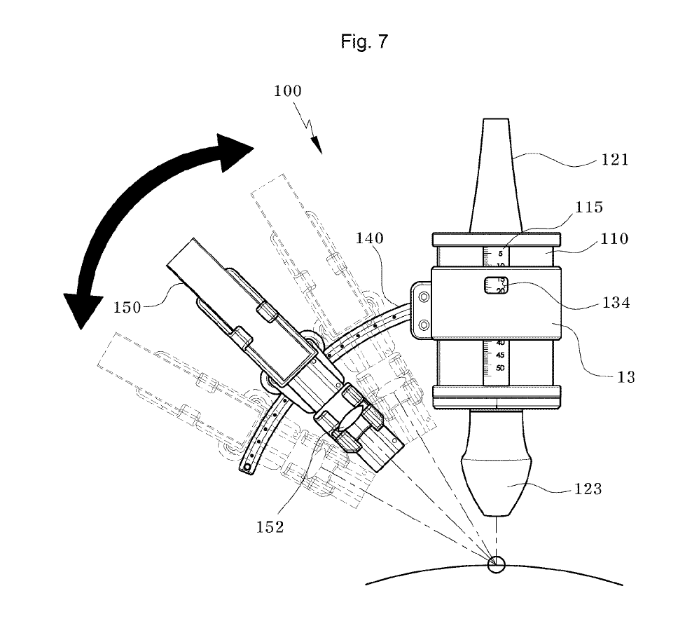

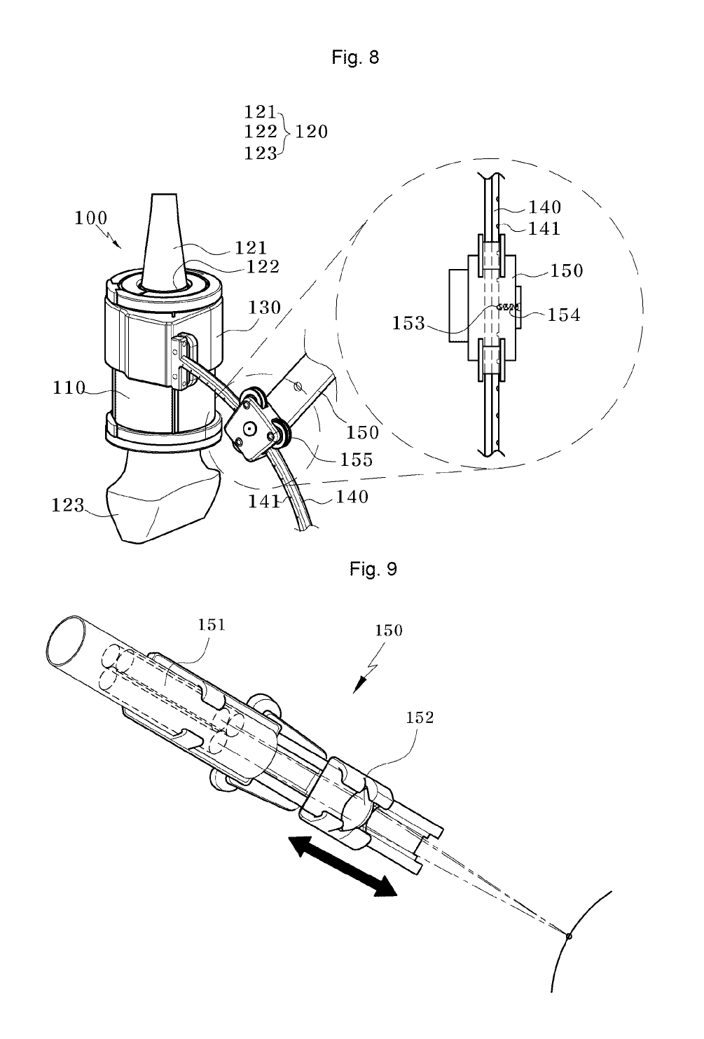

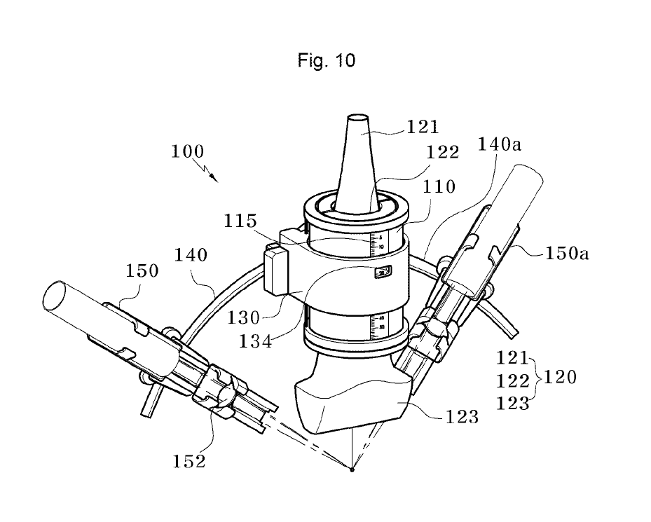

FIG. 7 is a view illustrating a first support and a first radiating unit of a laser treatment device using an ultrasonic probe according to the present invention. FIG. 8 is a perspective view illustrating a combination of a first support and a first radiating unit of a laser treatment device using an ultrasonic probe according to the present invention. FIG. 9 is a view illustrating a laser module and a convex lens of a laser treatment device using an ultrasonic probe according to the present invention. FIG. 10 is a view illustrating a second support and a second radiating unit of a laser treatment device using an ultrasonic probe according to the present invention.

Referring to FIG. 7, the first support 140 has one end connected with the elevator 130 and is shaped as an arc that has the same central axis along the lengthwise direction. The first radiating unit 150 is installed to be able to move along the lengthwise direction of the first support 140 to radiate laser beams. Specifically, the first radiating unit 150 has a plurality of moving wheels 155. The moving wheels 155 are provided on the top and bottom of the first support 140 to move along the first support 140.

Referring to FIG. 8, fastening holes 141 are formed in the rear surface of the first support 140 at constant intervals along the lengthwise direction of the first support 140. The first radiating unit 150 includes a fastener 153 that is fitted into the fastening hole 141 to fasten the first radiating unit 150 and an elastic body 154 that elastically supports the fastener 153 towards the fastening hole 141.

A combination of the first support 140 and the first radiating unit 150 is described. Where the first radiating unit 150 completes its movement while the fastener 153 is elastically supported by the elastic body 154 of the first radiating unit 150 towards the fastening hole 141, the fastener 153 is inserted to the fastening hole 141 by the elastic body 154, allowing the first radiating unit 150 to be fastened to the first support 140.

As such, there is no need for a separate means to fasten the first radiating unit 150 after the first radiating unit 150 is moved. Thus, the angle of the laser beam radiation from the first radiating unit 150 may be adjusted by simply moving the first radiating unit 150.

Referring to FIG. 9, the first radiating unit 150 includes a plurality of laser modules 151 radiating laser beams and a convex lens 152 focusing laser beams from the laser modules 151 onto the lesion. In this case, the convex lens 152 may be moved up or down to focus laser beams from the first radiating unit 150 onto the lesion.

According to the present invention, at least three or more laser modules 151 may be arranged at even intervals and may sequentially output and radiate laser beams. The sequential radiation of the laser beams from the laser modules 151 may prevent a surgical accident due to an error in the detecting the lesion.

According to the present invention, the laser treatment device 100 using an ultrasonic probe may adopt a remote-center-of-motion (RCM) mechanism so that the first radiating unit 140 may enable their laser beam radiation to reach the target despite a change in the angle of radiation due to its movement along the first support 140. Thus, the angle and depth to which laser beams are radiated are identified by the ultrasonic probe 120, and in this state, laser beams from the first radiating unit 150 may be radiated with their angle adjusted while avoiding any obstacle. In other words, although the angle of laser radiation is changed to avoid an obstacle, laser beams from the first radiating unit 150 may reach the target, i.e. lesion.

Referring to FIG. 10, according to the present invention, the laser treatment device 100 using an ultrasonic probe may further include a second support 140a and a second radiating unit 150a that have the same configuration as the first support 140 and the first radiating unit 150, respectively.

The second support 140a may be provided in an opposite position of the first support 140. Preferably, the second support 140a may be provided in a position turned at 90 degrees or 180 degrees from the first support 140 and the housing 110.

Thus, upon surgery, laser beams from the first radiating unit 150 and laser beams from the second radiating unit 150a may jointly be used, and the angle of laser radiation as per the position may be adjusted by moving the first support 140 and the second support 140a.

FIG. 11 is a perspective view illustrating a guide rail of a laser treatment device using an ultrasonic probe according to the present invention.

Referring to FIG. 11, the housing 110 includes an upper protrusion 111, a lower protrusion 112, and a guide rail 113 formed between the upper protrusion 111 and the lower protrusion 112 to guide the elevator 130 up or down.

There may be provided at least two or more guide rails 113. The elevator 130 may be slid up or down along the housing 110 by the guide rails 113 stably without rotation.

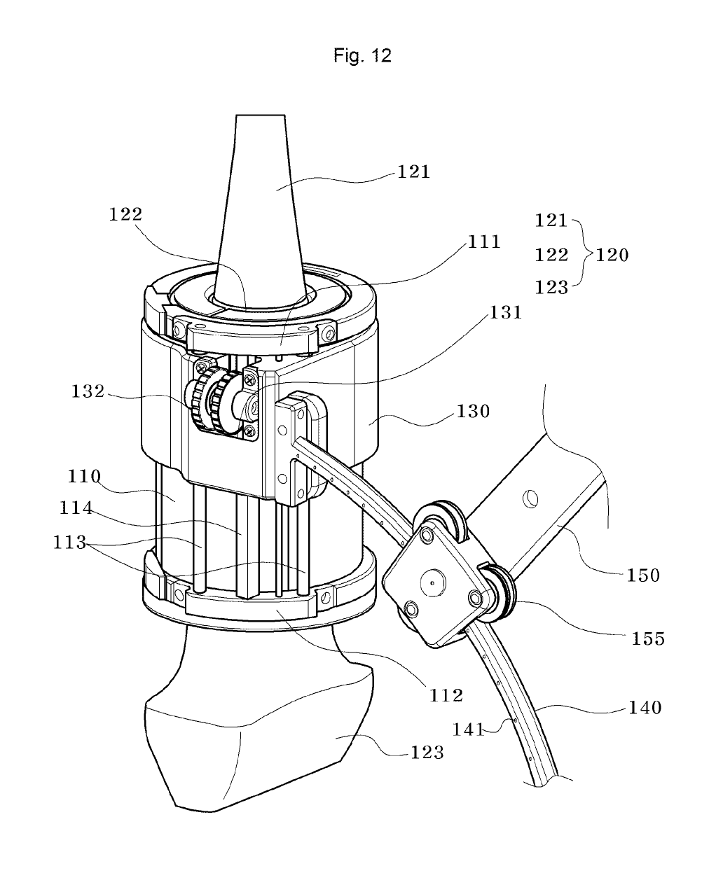

FIG. 12 is a perspective view illustrating a conveying rail, an opening, and an adjusting gear of a laser treatment device using an ultrasonic probe according to the present invention.

Referring to FIG. 12, the housing 110 further includes a conveying rail 114 formed between the upper protrusion 111 and the lower protrusion 112. The elevator 130 further includes an opening 131 formed in a position corresponding to the conveying rail 114, and adjusting gears 132 provided in the opening 131 to face the conveying rail 114 and rotating to allow the elevator 130 to slide along the conveying rail 114.

The adjusting gears 132 provided in the opening 131 are rotated along the conveying rail 132 to slide the elevator 130 up or down along the housing 110 and may be guided by the guide rails 113, allowing for a more stable slide.

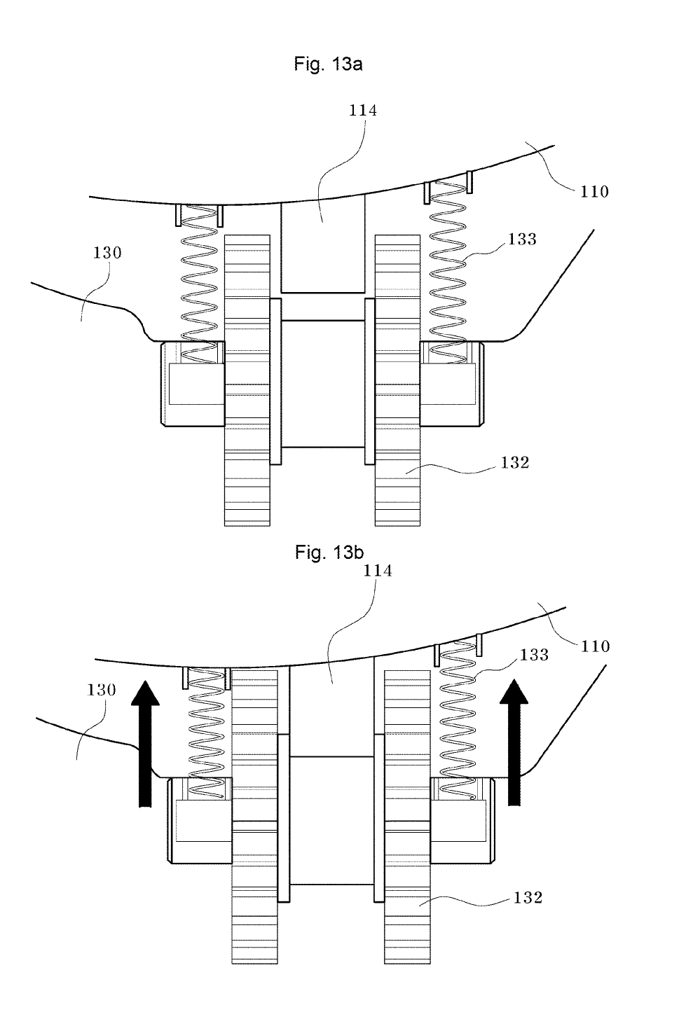

FIGS. 13a and 13b are perspective views illustrating elastic bodies of a laser treatment device using an ultrasonic probe according to the present invention.

Referring to FIGS. 13a and 13b, the elevator 130 further includes an elastic body 133 that is provided between the conveying rail 114 and the adjusting gears 132 to elastically support the adjusting gears 132 in a direction of spacing the adjusting gears 132 apart from the conveying rail 114.

In this case, the elastic body 133 may be formed with the same configuration as the elastic body 154 of the first radiating unit 150.

According to the present invention, where a force is applied to the adjusting gears 132 with the adjusting gears 132 elastically supported by the elastic body 133 of the elevator 130 in the direction of being spaced apart from the conveying rail 114, the interval between the conveying rail 114 and the adjusting gears 132 supported by the elastic body 133 may be reduced, and as the adjusting gears 132 rotate, the elevator 130 may be slid up or down along the housing 110.

Thus, although the adjusting gears 132 are mistakenly rotated in the state of the depth of radio radiation from the first radiating unit 150 having been completely adjusted, as long as no force is applied to the adjusting gears 132, the elastic body 133 may elastically support the adjusting gears 132, preventing a change from being made to the depth of laser radiation from the first radiating unit 150 and hence allowing for precise radiation to the lesion of laser beams from the first radiating unit 150.

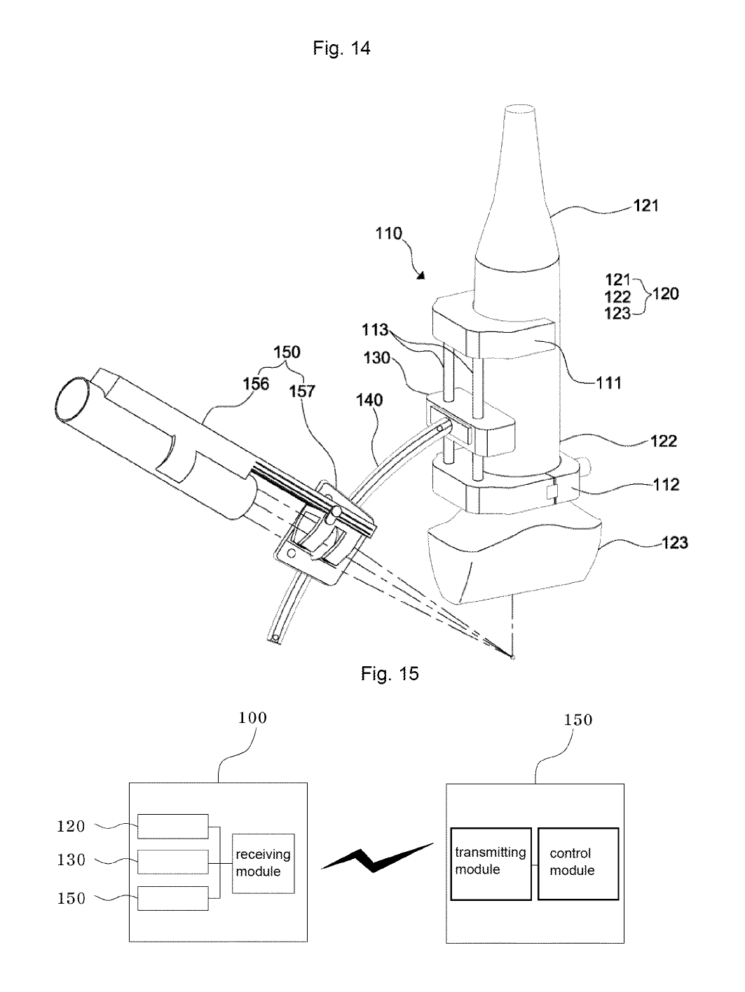

FIG. 14 is a perspective view illustrating another embodiment of an elevator and a first radiating unit of a laser treatment device using an ultrasonic probe according to the present invention.

Referring to FIG. 14, in the laser treatment device 100 using an ultrasonic probe, the elevator 130 is combined with the guide rails 113 formed between the upper protrusion 111 and lower protrusion 112 of the housing 110 and are rendered to ascend or descend. Although FIG. 14 illustrates an example in which the elevator 130 is shaped substantially as a hexahedron on one side of the body 122 of the ultrasonic probe 120, the elevator 130 may be shaped to surround the body 122 of the ultrasonic probe 120, which may be semi-circular in cross section, to be supported in a more stable manner.

According to the present invention, there may be at least two or more guide rails 113, and the elevator 130 may be combined with the two or more guide rails 113 and be slid up or down while being prevented from rotating with respect to the ultrasonic probe 120. At least one of the guide rails 113 may have a graduated unit to indicate the height of the elevator 130 as the elevator 130 slides.

In the laser treatment device 100 using an ultrasonic probe, according to the present invention, the first radiating unit 150 may include a radiating module 156 and a moving module 157. In the state of fastening the laser module 151 and the convex lens 152, the radiating module 156 may move up or down the convex lens 152 to allow laser beams from the laser module 151 to be focused. The moving module 157 includes a fastener 153, an elastic body 154, and moving wheels 155 and allows the first radiating unit 150 to move along the lengthwise direction of the first support 140.

In the first radiating unit 150, the moving module 157 may be provided on the rear surface of the radiating module 156 as shown in FIGS. 3 to 13 or on one side of the radiating module 156 as shown in FIG. 14.

Where the moving module 157 is provided on one side of the radiating module 156, the operator may have a broader vision that would otherwise be partially hidden by the radiating module 156.

FIG. 15 is a view illustrating an example of remotely controlling a laser treatment device using an ultrasonic probe according to the present invention.

According to the present invention, a laser treatment device 100 using an ultrasonic probe may include a controller 160 for remotely controlling a rotation of the ultrasonic probe 120, a slide of the elevator 130, and a movement of the first radiating unit 150.

Referring to FIG. 15, where the laser treatment device 100 using an ultrasonic probe has a receiving module, if a control signal produced from a control module of the controller 160 is transmitted through a transmitting module of the controller 160, the receiving module may receive the control signal to remotely drive the ultrasonic probe 120, the elevator 130, and the first radiating unit 150.

The present invention may detect a lesion using the ultrasonic probe and treat the legion by laser radiation, allowing for time savings for surgery. The present invention enables precise focusing only onto the legion upon treatment. The present invention allows laser beams from the first radiating unit to reach the lesion regardless of a change in the angle of laser radiation, reducing an error in the lesion.

While the present invention has been shown and described with reference to exemplary embodiments thereof, it will be apparent to those of ordinary skill in the art that various changes in form and detail may be made thereto without departing from the spirit and scope of the present invention as defined by the following claims.

According to the present invention, there may be provided a laser treatment device capable of detecting a lesion using an ultrasonic probe and radiating a laser beam to the lesion to treat the lesion, which has useful applications in this field.

What is claimed is:

1. A laser treatment device using an ultrasonic probe, comprising:
a housing 110;
the ultrasonic probe 120 including a knob 121, a body 122 connected to the knob 121, and a dispersing unit 123 connected to the body 122, the body 122 rotatably installed inside the housing 110;
an elevator 130 provided on an outer circumference of the housing 110 to slide up or down;
a first support 140 having an end connected to a side of the elevator 130 and shaped as an arc to have the same central axis along a lengthwise direction thereof; and
a first radiating unit 150 movably installed along a lengthwise direction of the first support 140 to radiate laser beams.

2. The laser treatment device of claim 1, wherein the first radiating unit 150 includes a plurality of laser modules 151 for radiating laser beams and a convex lens 152 focusing the laser beams radiated from the plurality of laser modules 151.

3. The laser treatment device of claim 2, wherein the number of the plurality of laser modules 151 is at least three or more, and the plurality of laser modules 151 are arranged at even intervals and sequentially output and radiate the laser beams.
